(19) United States
(12) Patent Application Publication
(10) Pub. No.: US 2004/0081962 A1
(43) Pub. Date: Apr. 29, 2004

(54) Methods for synthesizing complementary DNA

(76) Inventors: Caifu Chen, Palo Alto, CA (US); Ben Schroeder, San Mateo, CA (US); John Brandis, Hercules, CA (US); Gary Schroth, San Ramon, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/278,760

(22) Filed: Oct. 23, 2002

(51) Int. Cl. C12Q 1/68; C12P 19/34

(52) U.S. Cl. 435/6; 435/91.2

(57) ABSTRACT

The invention provides methods and kits for cDNA synthesis and for RNA amplification.

### FIELD OF THE INVENTION

The invention relates to nucleic acid amplification and methods for synthesizing cDNA by randomly priming second strand cDNA synthesis. The method can generate double stranded cDNA containing a site recognized by an RNA polymerase that can serve as a template for amplification by in vitro transcription.

### BACKGROUND OF THE INVENTION

The process of enzymatic conversion of poly(A)+ mRNA to double-stranded cDNA has become a key step for many molecular techniques. Since the first complementary DNA (cDNA) clones were obtained in the mid-1970s, several different methods have been developed to synthesize double-strand of cDNA from mRNA or from total RNA.

Synthesis of the first strand of cDNA generally involves use of RNA-dependent DNA polymerase (reverse transcriptase) to catalyze the reaction. Many different forms of reverse transcriptase are available commercially, including: the avian retrovirus reverse transcriptase, which is purified from particles of an avian retrovirus such as Avian myeloblastosis virus (AMV); the rTth DNA Polymerase, which is an ultra-pure, thermostable recombinant DNA polymerase blend of Thermus thermophilus (Tth) and Thermococcus litoralis (Tli) DNA polymerases; and the murine reverse transcriptase, which is a recombinant form of the reverse transcriptase from moloney murine leukemia virus (M-MLV or MuLV). The most common commercial reverse transcriptase is MuLV. One of skill in the art may choose a MuLV reverse transcriptase that has RNase H activity or that does not have RNase H activity (e.g. SuperScript II).

In general, two methods are employed to synthesize the second strand of cDNA preparations. One method employs self-priming. For reasons that are not fully understood, the 3′ termini of single-stranded cDNAs are capable of forming hairpin structures that can be used to prime the synthesis of the second cDNA strand by the Klenow fragment of E. coli DNA polymerase I or by the reverse transcriptase. In the presence of reverse transcriptase and dNTPs, dT₁₂₋₂₄ primers will prime DNA synthesis from the poly(A) tail of each transcript to generate a cDNA/mRNA hybrid. To allow hairpin structures to form, it is necessary to denature the hybrid molecules. The second strand cDNA can then be synthesized from the hairpin by adding Klenow DNA polymerase and dNTPs. The product of the self-primed synthesis of the second strand is a double-stranded cDNA molecule containing a loop at the end of the molecule. This loop is then digested with the single-strand-specific nuclease S1 to give a double strand cDNA molecule. In general, self-primed synthesis of the second strand of cDNA is a poorly controlled reaction, and subsequent cleavage of the hairpin structure often leads to the loss of the 5′ terminus of the mRNA (Land, Grez et al. 1981). Consequently, this self-priming scheme is not a method of choice.

Another method for priming second strand synthesis involves the use of RNase H. This method was introduced by Okayama et al. (1982) and Okayama et al. (1992) and then modified by Gubler and Hoffman (1983). The cDNA/mRNA product of first-strand synthesis is treated with RNase H to generate nicks and gaps in the hybridized mRNA. E. coli DNA polymerase I synthesizes the second strand of cDNA by starting within these nicks and gaps.

However, the currently available methods for making cDNA molecules still suffer from problems such as low yield and often do not generate populations or libraries that are representative of the original population of mRNA. For a variety of reasons, the low abundance mRNAs are sometimes lost or underrepresented in the final library. Hence, new methods for making cDNA molecules and for amplifying a population of RNA molecules are needed.

### SUMMARY OF CERTAIN EMBODIMENTS OF THE INVENTION

The invention provides an improved cDNA synthesis method for efficiently generating substantially full-length cDNA populations. The methods of the invention can produce cDNA populations that can have reduced bias toward synthesis of certain sizes and concentrations of RNA, for example, when compared to the RNaseH method of generating cDNA. Such methods can be adapted to generate useful quantities of amplified cRNA product that comprises a population of cRNA molecules in substantially the same relative molar ratio as the RNA or mRNA starting material.

One aspect of the invention therefore provides a method for synthesizing a double-stranded cDNA comprising: (a) synthesizing a pool of first cDNA strands in a first reaction mixture comprising reverse transcriptase, a template RNA population and a mixture of primers that are complementary to at least a portion of the template RNA population; (b) removing the template RNA population; and (c) synthesizing a pool of double stranded cDNAs in a second reaction mixture comprising a processive DNA polymerase (e.g. from Bacillus stearothermophilus, Bst), DNA ligase, the first cDNA strand as template and multiple random primers, wherein the multiple random primers comprise a mixture of oligonucleotides having random DNA sequences.

The mixture of primers used for first strand synthesis can be oligo dT-RNAP primers, each comprising a single stranded oligonucleotide with a first segment and a second segment, wherein the first segment comprises about 10 to about 30 deoxyribothymidine nucleotides and the second segment comprises a recognition site for an RNA polymerase.

In another embodiment, the invention provides a method for amplifying a population of RNA molecules, comprising: (a) synthesizing a pool of first cDNA strands in a first reaction mixture comprising reverse transcriptase, a template RNA population and a mixture of primers that are complementary to at least a portion of the RNA molecules in the template RNA population, to produce a pool of first cDNA strands; (b) removing the template RNA population; (c) synthesizing a pool of double stranded cDNAs in a second reaction mixture comprising the pool of first cDNA strands as template, a processive DNA polymerase, DNA ligase, and multiple random primers, to produce a pool of double-stranded cDNA molecules; and (d) synthesizing an amplified complementary RNA population in a third reaction mixture comprising an RNA polymerase and the pool of double-stranded cDNA molecules as template, to produce an amplified complementary RNA population having about the same proportion of each RNA type as in the template RNA population of step (a). The mixture of primers can be oligo dT-RNAP primers that comprise a pool of single stranded oligonucleotides each having a first segment and a second segment, wherein the first segment comprises about 10 to about 30 deoxyribothymidine nucleotides and the second segment comprises a recognition site for a RNA polymerase.

The template RNA population can have one or more low abundance mRNAs and one or more high abundance mRNAs. The processive DNA polymerase can be Bacillus stearothermophilus DNA Polymerase. Examples of RNA polymerases that can be used in the methods of the invention are T7 RNA polymerase, T3 RNA polymerase and SP6 RNA polymerase. One type of oligo dT-RNAP primer provided by the invention comprises SEQ ID NO:1. The reverse transcriptase can be, for example, moloney murine leukemia virus reverse transcriptase. The first and second reaction mixtures can also include a mixture of deoxyribonucleoside triphosphates. The third reaction mixture can include a mixture of ribonucleoside triphosphates.

In one embodiment, the invention provides a kit for synthesizing a double-stranded cDNA comprising a first container containing a processive DNA polymerase (for example, a Bacillus stearothermophilus DNA polymerase) and a second container containing multiple random primers, wherein the multiple random primers comprise a mixture of oligonucleotides having random DNA sequences. The kit can further comprise a third container containing a mixture of primers for first cDNA strand synthesis. Such first cDNA strand synthesis primers are complementary to a portion of at least some of the RNA molecules in the template RNA population. An example of one type of first cDNA strand primers is the oligo dT-RNAP primers provided herein, wherein the oligo dT-RNAP primers comprise a pool of single stranded oligonucleotides each having a first segment and a second segment, wherein the first segment comprises about 10 to about 30 deoxythymidine nucleotides and the second segment comprises a recognition site for a RNA polymerase.

In another embodiment, the invention provides a kit for amplifying a population of mRNA molecules, comprising a first container containing a Bacillus stearothermophilus DNA polymerase, a second container containing multiple random primers, wherein the multiple random primers comprise a mixture of oligonucleotides having random DNA sequences; and a third container containing oligo dT-RNAP primers, wherein the oligo dT-RNAP primers comprise a pool of single stranded oligonucleotides each having a first segment and a second segment, wherein the first segment comprises about 10 to about 30 deoxythymidine nucleotides and the second segment comprises a recognition site for a RNA polymerase. One RNA polymerase that can be used in the kits of the invention is T7 RNA polymerase. An example of an oligo dT-RNAP primer is a primer having SEQ ID NO:1. The kit can also include a container containing a reverse transcriptase, a container containing a DNA ligase, a container containing a mixture of deoxyribonucleoside triphosphates, a container containing a mixture of ribonucleoside triphosphates, a container containing a labeled deoxyribonucleoside triphosphate, or a container containing a labeled ribonucleoside triphosphate. The kits of the invention can also contain instructions for making cDNA or instructions for amplifying a population of mRNA molecules.

### DESCRIPTION OF THE FIGURES

FIG. 1 provides a schematic diagram illustrating the steps involved in second strand cDNA synthesis using a self-priming method. In the first step, a cDNA:RNA hybrid is created by first strand synthesis of a cDNA on an RNA template. A poly-A tail (“AAn”) is present on the RNA template. The cDNA strand in this diagram was generated using an oligo dT-RNAP primer (identified as “TT-T7” on the end of the cDNA strand). After first strand synthesis, the cDNA:RNA hybrid is heat denatured and/or the RNA is hydrolyzed with base. The single-stranded cDNA can fold back on itself at one end to effectively act as a primer (with a free 3′-hydroxyl) for synthesis of a double-stranded cDNA strand when dNTPs and DNA polymerase is added. A double-stranded cDNA with a hairpin loop is then formed. Digestion with S1 nuclease can provide a double-stranded cDNA without the hairpin loop.

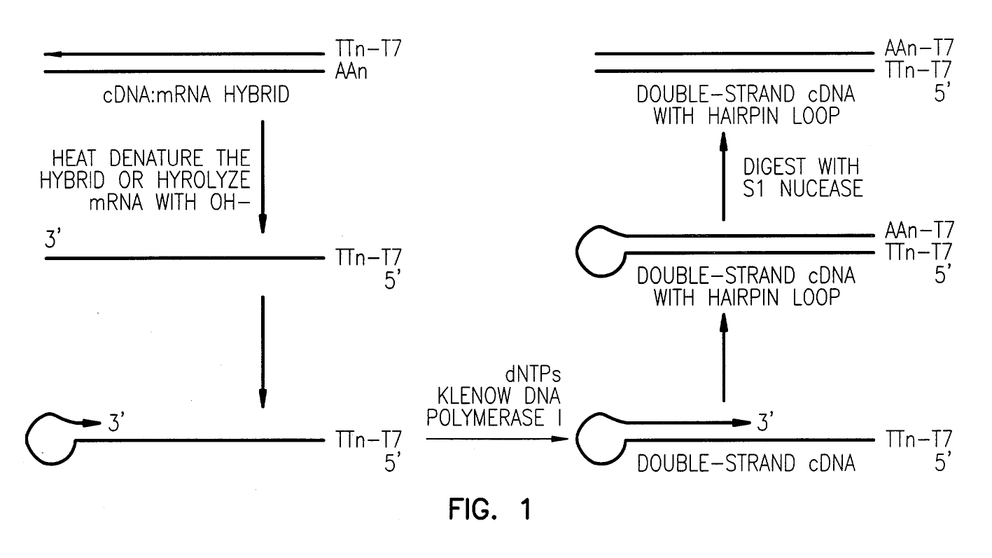

FIG. 2 provides a schematic diagram illustrating the steps involved in second strand cDNA synthesis using an RNase H method. In the first step, reverse transcriptase (“RT”) is used to synthesize a cDNA:RNA hybrid. A poly-A tail (“AAAAAn”) is present on the RNA template. The cDNA strand in this diagram was generated using an oligo dT-RNAP primer (identified as “T7-TTTTTn”). RNase H is used to nick portions of the RNA template. Then, DNA polymerase and DNA ligase are used to generate a second cDNA strand, thereby forming a double-stranded cDNA product (ds-cDNA).

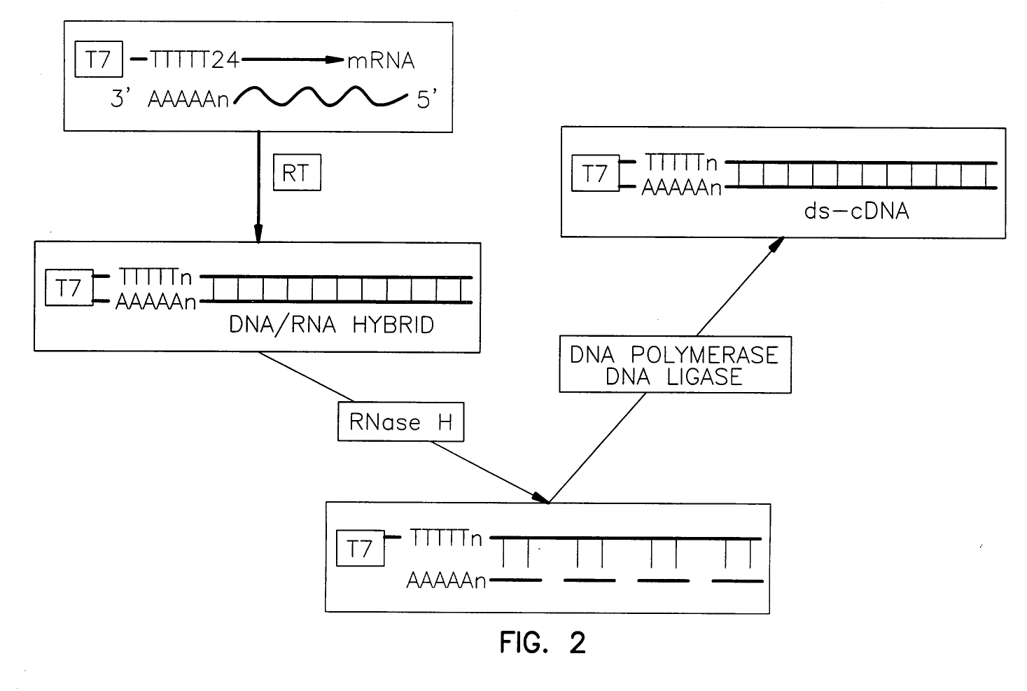

FIG. 3 provides a schematic diagram illustrating the steps involved in second strand cDNA synthesis using the random priming method of one embodiment of the invention. In the first step, reverse transcriptase (“RT”) is used to synthesize the first strand of the cDNA, thereby forming a cDNA:RNA hybrid. A poly-A tail (“AAAAAn”) is present on the RNA template provided in this diagram. The cDNA strand in this diagram was generated using an oligo dT-RNAP primer (identified as “T7-TTTTT24”). After formation of a cDNA/RNA hybrid, the RNA template is hydrolyzed using base (e.g. 0.3 M NAOH). The first strand is single-stranded at this point. Then DNA polymerase (“DNA Pol”), ligase and random primers (“pN₈₋₉”) are used to generate the second strand. A double-stranded cDNA product (“ds-cDNA”) is thereby formed.

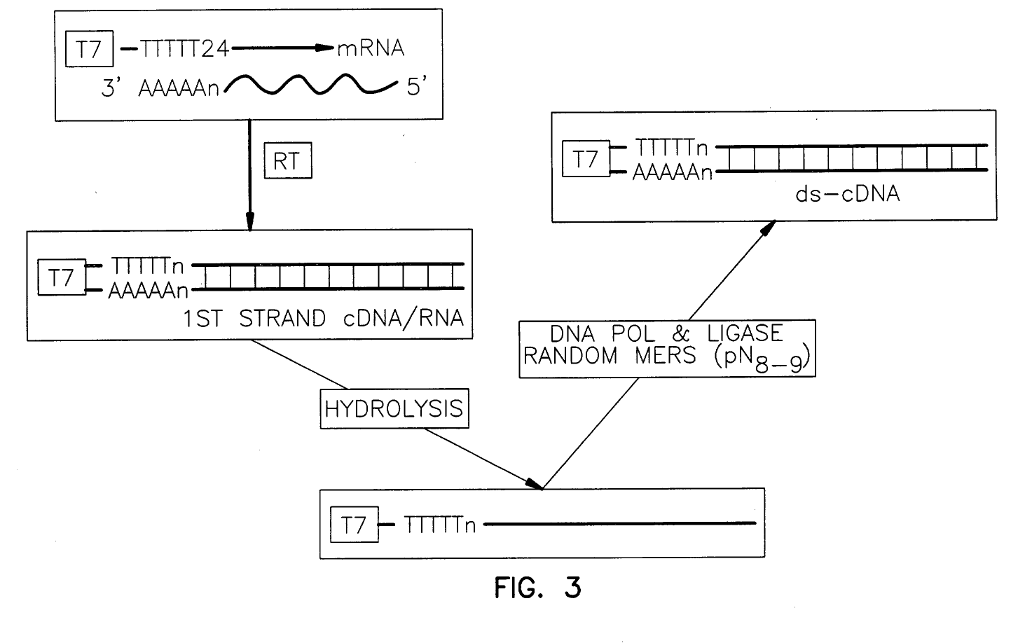

FIG. 4 is a photograph of a 2% agarose gel illustrating the size and amounts of cRNA product generated using the RNase H method or a non-optimized random priming method for second strand synthesis. Lane M provides molecular weight markers. Lane 1 shows the product of an RNase H method where Superscript II reverse transcriptase was used for first strand synthesis. Lane 2 shows the product of an RNase H method where MuLV reverse transcriptase was used for first strand synthesis. Lane 3 shows the cRNA product obtained after amplification of cDNA generated using non-optimized random priming second strand synthesis.

FIG. 5 shows the cRNA products generated by amplification of cDNA produced using a variety of temperatures and a variety of DNA polymerases for the random priming method. The type of second strand synthesis method (random priming or RNase H), the type of polymerase (T4, Bst or E. coli) and the temperature of the second strand synthesis reaction (20° C., 25° C. or 37° C.) are provided at the top. After cDNA synthesis and amplification by in vitro RNA synthesis, each cRNA product was column purified and then visualized on a 2% agarose gel. As illustrated, the random priming method produces the most cRNA product when performed at 37° C. using Bacillus stearothermophilus (Bst) DNA polymerase. Also as illustrated, the cRNA product was substantially full length.

### DETAILED DECRIPTION OF EMBODIMENTS OF THE INVENTION

The invention provides a method for synthesizing a double-stranded cDNA. The method involves synthesizing a pool of first cDNA strands in a first reaction mixture, removing the RNA template, and then synthesizing a pool of double stranded cDNAs using a processive DNA polymerase and multiple random primers. The first reaction mixture can include a mixture of reverse transcriptase, one or more RNA templates and a primer that is complementary to at least one RNA template. The primer can include a stretch of poly(dT) nucleotides for priming synthesis from a poly(A) mRNA tail. The dT primer can also encode a promoter for synthesis of complementary RNA. The multiple random primers used for second strand synthesis generally comprise a mixture of oligonucleotides having random DNA sequences.

Definitions

Unless defined otherwise, all technical and scientific terms used herein have the same meaning as commonly understood by one of ordinary skill in the art to which this invention belongs. A few terms are further discussed below.

The singular forms “a,” “an” and “the” include plural referents unless the context clearly dictates otherwise. Thus, for example, reference to “a primer” means that more than one such primer can be present in the composition.

The term cRNA refers to a complementary RNA that is generated by in vitro RNA transcription of a cDNA. Such a cRNA can be produced by the methods of the invention, for example, when a first strand synthesis primer is used that encodes an RNA promoter. Such an RNA promoter is then incorporated into the cDNA. Mixing an appropriate RNA polymerase with such a cDNA under conditions suitable for RNA transcription leads to synthesis of cRNA.

An oligo dT-RNAP primer comprises a pool of single stranded oligonucleotides. Each oligonucleotide in the oligo dT-RNAP primer pool has a first segment and a second segment, wherein the first segment comprises about 10 to about 30 dT nucleotides and the second segment comprises a recognition site for an RNA polymerase. The recognition site for an RNA polymerase is a sequence that can function as a promoter. Examples of RNA polymerases that may be used in the invention include, for example, T3, SP6 and T7 RNA polymerases.

As used herein, the term “primers” means short nucleic acids, whether occurring naturally as in a purified restriction digest or produced synthetically, which may be annealed to a target polynucleotide by complementary base-pairing. After annealing, a primer may be extended along the target RNA or DNA strand by a reverse transcriptase or by a DNA polymerase enzyme. Primers can be made from natural or modified deoxyribonucleotides, ribonucleotides or analogs thereof.

The terms “complementary” and “complementarity” refer to the natural binding of polynucleotides by hydrogen bond base pairing. For example, complementary binding can occur between the sequence 5′ A-G-T 3′ and the complementary sequence 3′ T-C-A 5′. Complementarity between two single-stranded molecules may be “partial,” such that only some of the nucleotides in the two nucleic acids bind, or it may be “complete,” such that total complementarity exists between the single stranded molecules. The degree of complementarity between nucleic acid strands significantly affects the efficiency and strength of the hybridization between the nucleic acid strands.

The phrase “removing the RNA template” means that substantially all of the RNA template is dissociated and separated from a synthesized cDNA so that substantially no RNA template is present during second strand synthesis. The RNA template can be removed by any available means. In some embodiments, the RNA template is physically removed, for example, by purification of the cDNA away from the RNA template using affinity separation or phase separation. Alternatively, in other embodiments the RNA template is removed by chemical means, for example, by alkaline hydrolysis.

Methods

In certain embodiments, the methods of the invention are used to synthesize a double-stranded cDNA. These methods involve synthesizing the first cDNA strand of selected RNA molecules in an RNA sample (e.g. the mRNA template population), removing the RNA template, and generating a pool of double stranded cDNAs using a DNA polymerase and multiple random primers. The multiple random primers generally comprise a mixture of oligonucleotides having random DNA sequences. Details of these procedures are provided below.

The cDNA produced by the methods of the invention can be amplified to generate a useful pool of cDNA or cRNA molecules. In one embodiment, the invention provides a method for amplifying a population of mRNA molecules. Such a method involves synthesizing a pool of first cDNA strands, removing the template RNA population, synthesizing a pool of double stranded cDNAs and synthesizing an amplified cRNA population. The primer used for synthesizing the pool of first cDNA strands can be any primer complementary to at least a portion of the RNA molecules in the RNA template population. For example, the first strand cDNA primer can be an oligo dT-RNAP primer that includes a pool of single stranded oligonucleotides, each having a first segment and a second segment, wherein the first segment comprises about 10 to about 30 dT nucleotides and the second segment comprises a recognition site (i.e. a sequence that can function as a promoter) for an RNA polymerase. Such an RNA Polymerase can be, but is not limited to, a T3, SP6 or T7 RNA polymerase. The cRNA generated by transcription from the promoter may optionally be labeled by incorporation of labeled nucleotides, e.g. fluorescent dye labeled ribonucleotides, biotin labeled ribonucleotides, digoxigenen labeled ribonucleotides, and the like.

The cDNA and cRNA products generated by some of the methods of the invention can be used to determine quantitative information about the genetic profile of the nucleic acids in the original RNA sample, as well as the physiological source from which the labeled sample nucleic acid was derived. The data provides information about the physiological source from which the sample nucleic acid were derived, such as the types of genes expressed in the tissue or cell which is the physiological source, as well as the levels of expression of each gene, in quantitative or quantitative terms.

The methods of the invention can be used in comparing nucleic acid samples from two or more physiological sources to identify and quantify differences between the mRNAs in the sources and thereby provide data on the differential expression of a particular gene in the physiological sources being compared. The methods of the invention find use in differential gene expression assays for the analysis of a diseased and normal tissue, analysis of a different tissue or sub-tissue types, and the like. The methods of the invention may be used for large-scale correlation studies on the sequences, mutations, variants, or polymorphisms among samples.

RNA Samples

Samples to be used in the methods of the invention are from any source identified as containing, or expected to contain, RNA. One of skill in the art may merely suspect or expect a sample to contain one or more RNA molecules of interest. For example, samples can be body fluids (e.g., blood, urine, saliva, mucus), tissue samples, cultured prokaryotic or eukaryotic cells, environmental samples (for example, soil or water) and the like. Thus, for example, body fluid or tissue samples can be obtained from a patient or other animal for examining or studying the expression patterns or types of mRNAs in the patient or animal. The patient or animal may be infected or suffering from a disease condition and thus be screened for an infective agent or gene that may be responsible for the disease condition. The sample can be an environmental sample (for example, soil or water) suspected of harboring a particular organism that is screened to determine which samples contain a particular RNA indicative of the organism. Moreover, a sample can be from any source containing or suspected of containing nucleic acid, where the nucleic acid has at least some RNA.

For example, the sample may be a human, animal or veterinary clinical specimen. The sample may be from an agricultural or food product. The sample can be a biological fluid such as plasma, serum, blood, urine, sputum or the like. The sample may contain bacteria, yeast, viruses and the cells or tissues of higher organisms such as plants or animals, suspected of harboring an RNA of interest.

The source of nucleic acid is RNA in purified or non-purified form. Samples can be manipulated by procedures available in the art so that the samples are suitable for use in the methods of the invention, or in other methods of nucleic acid detection. Methods for the extraction and/or purification of RNA are available to one of skill in the art, for example, by Maniatis et al., Molecular Cloning: A Laboratory Manual (New York, Cold Spring Harbor Laboratory, 1982); Sambrook et al., Molecular Cloning: A Laboratory Manual, 2^{nd} Edition (1989); Sambrook and Russell, Molecular Cloning: A Laboratory Manual, 3^{rd} Edition (Jan. 15, 2001).

RNA Sources

Certain embodiments of the disclosed methods can be used to detect or amplify any RNA molecule or combination of RNA molecules in a sample. RNA molecules for detection, referred to herein as RNA molecules of interest, or target RNA molecules, are selected based on the needs and purpose of one of skill in the art. For example, a particular RNA molecule may be associated with a particular cell type, pathogen, disease state, or predisposition to a disease. Detection of such an RNA molecule may or may not have a diagnostic value. For example, mRNA can be detected or amplified that is specific to particular tumor cell type or to a particular normal cell type.

Certain embodiments of the disclosed methods can also be used to determine the ratio of expression of different RNA species from individual organisms or an individual sample. For this purpose, the method is used to detect multiple RNA species simultaneously. Arrays of probes for RNA species are useful for detection of such multiple RNA species. The methods of the invention can also be used to detect similar or related RNAs where the related RNAs have a common sequence motif between them, but where the RNAs are otherwise different. For example, cells may contain multiple RNA species having similar regulatory sequences, similar structural motifs, or other sequences in common. Such classes of RNA molecules can be detected with a single primer species by designing a primer to hybridize to the common sequence.

Certain embodiments of the disclosed methods can also be used to detect a plurality of different RNA molecules of interest in a sample from an individual organism. This is accomplished, for example, by using a selected primer to screen for an RNA sequence that is present in each of the RNA molecules of interest, or by screening with multiple primers that are collectively complementary to one or more regions on each of the RNA molecules of interest. The later approach can be used for detecting, for example, some diseases or predispositions to disease that are associated with different mutations in particular genes. In such cases, one of skill in the art may screen for sequences complementary to regions characteristic of each mutation within a mutant RNA.

The methods of the invention can also be used to detect one or more RNAs of interest that are members of a gene family. The gene family may be from one type of organism (e.g. humans) or may be present in different organisms, such as in fungi, bacteria, plants, animals and viruses. Substantially all members of a gene family may be detected and amplified by utilizing a first strand primer that anneals to a sequence that is conserved or shared by substantially all members of the gene family. Any conserved nucleic acid sequence may be utilized for this purpose. For example, to detect mycobacteria, one of skill in the art may utilize primers that selectively hybridize to an RNA molecule that is both unique to mycobacteria and present in most types of mycobacteria.

RNA molecules of interest for use in the disclosed method can come from a variety of natural or synthetic sources, for example, the RNA can be messenger RNA, ribosomal RNA, nucleolar RNA, transfer RNA, viral RNA, heterogeneous nuclear RNA, or the like. In addition, naturally occurring or synthetic fragments of RNA molecules may be detected. The identification and selection of nucleic acid molecules for detection is a well-developed area and such identification and selection criteria can be fully applied to the identification and selection of RNA molecules as targets of detection in the disclosed method.

Once a source of RNA and/or one or more RNAs of interest have been identified, a primer is needed to begin the reverse transcription. In the methods of the invention, two general types of primers are used: a primer for first strand synthesis and a collection of short primers having random sequences for second strand synthesis. Such primers may be adapted as desired by one of skill in the art without departing from the scope and teachings of the invention.

Primers

First and second strand primers are used for synthesis of the first and second strands of a cDNA. Such primers are oligonucleotides that comprise nucleotides. The term “nucleotide” is used herein to mean an individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications such as (a) an alternative linking group, (b) an analogous form of purine, (c) an analogous form of pyrimidine, or (d) an analogous sugar.

Examples of analogous linking groups, purine, pyrimidines, and sugars can be found, for example, in PCT publication No. WO 95/04064, which disclosure is hereby incorporated by reference in its entirety. Modified nucleotides include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethyl-aminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxy-methyluracil, 5-methoxyuracil, 2-methylthio-N-6-isopenten-yladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxyp-propyl) uracil, and 2,6-diaminopurine.

Primers can also be conjugated to any detectable label. Such labels and methods for attaching labels to deoxyribonucleotides or ribonucleotides are available in the art. Labels include radionuclides, fluorescent dyes, enzymes that provide a detectable product upon exposure to a suitable substrate, and the like. Primers can be phosphorylated, for example, on the 5′ end.

The primers used in the methods of the invention may be prepared by any known method, including synthetic, recombinant, ex vivo generation, or a combination thereof, as well as utilizing any purification methods known in the art.

In one embodiment primers for use in the disclosed method are synthesized using established oligonucleotide synthesis methods. Methods to produce or synthesize oligonucleotides are available in the art. Such methods range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABI Model 380 B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method).

Methylenemethylimino linked oligonucleosides as well as mixed backbone compounds having, may be prepared as described in U.S. Pat. Nos. 5,378,825; 5,386,023; 5,489,677; 5,602,240; and 5,610,289, which disclosures are hereby incorporated by reference in their entireties. Formacetal and thioformacetal linked oligonucleosides may be prepared as described in U.S. Pat. Nos. 5,264,562 and 5,264,564, which disclosures are hereby incorporated by reference in their entireties. Ethylene oxide linked oligonucleosides may be prepared as described in U.S. Pat. No. 5,223,618, which disclosure is hereby incorporated by reference in its entirety. Phosphinate oligonucleotides may be prepared as described in U.S. Pat. No. 5,508,270, which disclosure is hereby incorporated by reference in its entirety. Alkyl phosphonate oligonucleotides may be prepared as described in U.S. Pat. No. 4,469,863, which disclosure is hereby incorporated by reference in its entirety. 3′-Deoxy-3′-methylene phosphonate oligonucleotides may be prepared as described in U.S. Pat. Nos. 5,610,289 or 5,625,050, which disclosures are hereby incorporated by reference in their entireties. Phosphoramidite oligonucleotides may be prepared as described in U.S. Pat. No. 5,256,775 or U.S. Pat. No. 5,366,878 which disclosures are hereby incorporated by reference in their entireties. Alkylphosphonothioate oligonucleotides may be prepared as described in published PCT applications WO 94/17093 and WO 94/02499 which disclosures are hereby incorporated by reference in their entireties. 3′-Deoxy-3′-amino phosphoramidate oligonucleotides may be prepared as described in U.S. Pat. No. 5,476,925, which disclosure is hereby incorporated by reference in its entirety. Phosphotriester oligonucleotides may be prepared as described in U.S. Pat. No. 5,023,243, which disclosure is hereby incorporated by reference in its entirety. Borano phosphate oligonucleotides may be prepared as described in U.S. Pat. Nos. 5,130,302 and 5,177,198 which disclosures are hereby incorporated by reference in their entireties.

Further details on primers utilized in the methods of the invention are provided below.

First Strand Synthesis

First strand synthesis involves generating a DNA strand that is complementary to an RNA of interest (cDNA). A reaction mixture that includes the RNA of interest, a complementary primer and reverse transcriptase is utilized to generate the first cDNA strand. The RNA sample or RNA of interest is selected by one of skill in the art, for example, as described herein.

The reverse transcriptase is any reverse transcriptase available to one of skill in the art, for example, Avian myeloblastosis virus (AMV); the rTth DNA Polymerase, which is an ultra-pure, thermostable recombinant DNA polymerase blend of Thermus thermophilus (Tth) and Thermococcus litoralis (Tli) DNA polymerases; or the murine reverse transcriptase, which is a recombinant form of the reverse transcriptase from moloney murine leukemia virus (M-MLV or MuLV). In one embodiment, the reverse transcriptase is from moloney murine leukemia virus.

The primer for first strand synthesis is an oligonucleotide having sequence complementary to a region on at least a portion of the RNA molecules in the template RNA population. Because mRNA molecules typically have a 3′ poly A tail, the primer employed often has region comprising a complementary oligo dT. For example, each of the primers in the mixture may contain 10 to 20 nucleotides that are capable of hybridizing to the polyA tail. However, the primer can also include other nucleotides that may or may not be complementary to an mRNA. Primers for first strand synthesis can have any sequence that is complementary to at least a portion of the RNA molecules in the template RNA population.

In certain embodiments, primers for first strand synthesis can contain two or more nucleotides at one end that can hybridize to the mRNA sequence that is immediately upstream of the polyA tail. The primers for first strand synthesis can contain a restriction endonuclease recognition sequence at one or the other end. The restriction enzyme sequence can be the same or different for all the primers in the mixture, and may be any restriction endonuclease recognition sequence known in the art. The primer for first strand synthesis can also include a promoter sequences. In one embodiment of the invention, the primer includes promoter sequences for T3 RNA Polymerase, SP6 RNA Polymerase or T7 RNA Polymerase. One example of a useful primer for first strand synthesis has SEQ ID NO:1 (CGAATTTAATACGACTCACTATAGGGAGATTTTTTTTTTTTTTTTTTTTTT TT).

A mixture of primers having slightly different sequences may be used to allow the synthesis of cDNA to begin accurately at the start of the polyA tail of the gene, for example, where the polyA tail is imperfect in that it may contain non-A nucleotides. The art method of preparing cDNA from mRNA usually result in about 60% successful reads when sequencing from the 3′ (polyA tail) end is attempted. In contrast, the use of the mixture of primers can result in a higher percentage, for example, in greater than 80%, successful reads.

Hybridization of the primer to the RNA molecule of interest can be carried out under any suitable conditions, and preferably under conditions that favor hybrid formation between RNA and DNA. See, for example, Sambrook et al., Molecular Cloning: A Laboratory Manual, 2nd Edition (Cold Spring Harbor Laboratory Press, Cold Spring Harbor, N.Y., 1989); Sambrook et al., Molecular Cloning: A Laboratory Manual, 2nd Edition (Cold Spring Harbor Laboratory Press, Cold Spring Harbor, N.Y., 2001).

Reverse transcription can be carried out using a reverse transcriptase, preferably a reverse transcriptase lacking RNase H function. The reaction mixture including the RNA molecule of interest, the primer, and the reverse transcriptase is then incubated under conditions to allow reverse transcription of the RNA molecule of interest and formation of DNA/RNA hybrids.

After reverse transcription the first cDNA strand can be purified to remove non-incorporated primer, nucleotides, enzymes and other components of the reverse transcription reaction that are no longer needed. The mRNA template can also be removed, for example, by denaturing or melting the RNA/cDNA hybrid and by physically separating the cDNA from the RNA (for example, by using solid phase separation), or by chemically destroying the RNA through alkaline hydrolysis.

In some embodiments, the purified first cDNA strand can be coupled to a solid support or substrate. Substrates for attachment to first strand cDNA molecules include any solid material to which the primers can be coupled or adhered. This includes materials such as acrylamide, cellulose, nitrocellulose, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates can have any form useful to one of skill in the art including thin films or membranes, beads, bottles, dishes, slides, fibers, woven fibers, shaped polymers, particles, chips and microparticles. Microtiter dishes, glass slides, chips and tagged beads can also be used as substrates.

First strand cDNAs immobilized on a substrate allow formation of double-stranded cDNA hybrids that are localized on the substrate. Such localization provides a convenient means of washing away reaction components that might interfere with subsequent amplification or detection steps. Attachment to a solid support may facilitate storage and further manipulation of cDNAs. Any method available to one of skill in the art can be used for immobilization of first strand cDNAs.

Second Strand Synthesis

Second strand synthesis is carried out using a reaction mixture that includes the first cDNA strand synthesized as described above, a population of random primers, and a processive DNA polymerase. This reaction mixture is then incubated under conditions to allow primer extension and formation of DNA/DNA hybrids.

A processive DNA polymerase is a DNA polymerase that remains associated with a template nucleic acid strand for a sufficient period of time to substantially extend an annealed primer and produce an extension product of significant length. Such processive DNA polymerases can be distinguished from non-processive DNA polymerases because processive DNA polymerases produce substantially full-length cDNA strands. Such processive DNA polymerases may also have good strand displacement activities, meaning that the DNA polymerase will preferentially continue extending a DNA strand it has begun synthesizing even though the template becomes double stranded. Non-processive DNA polymerases tend to dissociate from their template when they encounter a double-stranded segment in the template. In other embodiments, the DNA polymerases employed in the invention do not have substantial 3′ to 5′ exonuclease activity. In other embodiments, the processive DNA polymerase may not have substantial 5′ to 3′ exonuclease activity.

One example of a processive DNA polymerase is the DNA polymerase from Bacillus stearothermophilus (Bst). Such a Bst DNA polymerase can be a Bst DNA Polymerase Large Fragment that is the portion of the Bacillus stearothermophilus DNA Polymerase protein that contains the 5′->3′ polymerase activity, but lacks the 3′->5′ exonuclease domain. The Bst DNA polymerase has been used for DNA sequencing through high GC regions and for rapid sequencing using nanogram amounts of DNA template. One unit of Bst DNA polymerase can be defined as the amount of enzyme that will incorporate 10 nmol of dNTP into acid insoluble material in 30 minutes at 65° C. Such a Bst DNA polymerase is available from New England Biolabs.

Another example of highly processive DNA polymerases are the DNA polymerases encoded by bacteriophage Φ29 and similar bacteriophages described in, among other places, U.S. Pat. No. 5,576,204.

Conditions for use of DNA polymerases are available to one of skill in the art. For example, conditions for use of Bacillus stearothermophilus DNA polymerase are described in BioTechniques 30:852-867. The reaction buffer used for Bst DNA polymerase contains 20 mM Tris-HCl (pH 8.8), 10 mM KCl, 10 mM (NH4)₂SO₄, 2 mM MgSO₄, 0.1% Triton X-100. Bst DNA polymerase can be stored at −20° C. in 100 μg/ml BSA using 50 mM KCl, 10 mM Tris-HCl (pH 7.5), 1 mM dithiothreitol, 0.1 mM EDTA, 0.1% Triton X-100 and 50% glycerol. Reaction temperatures above 70° C. are not recommended for Bst DNA polymerase.

A second strand synthesis reaction using Bst DNA polymerase can be performed using, for example, 50 mM KCl, 20 mM Tris-HCl (pH 8.8), 10 mM MgCl₂, 30 nM first strand cDNA template, 70 nM random sequence primer, 200 μM dATP, 200 μM dCTP, 200 μM dGTP, 200 μM dTTP, 100 μg/ml BSA and Bst DNA polymerase with incubation at 65° C. After the reaction, Bst DNA polymerase can be heat inactivated at 80° C. for 10 minutes. See, Hugh, G. and Griffin, M. (1994) PCR Technology, p.p. 228-229; McClary, J. et al. (1991) J. DNA Sequencing and Mapping, p. 173-180; Mead, D. A. et al. (1991) BioTechniques, p. 76-87.

After second strand synthesis, the DNA/DNA hybrids formed may have nicks. Such nicks can be ligated by a convenient ligase, for example, T4 DNA ligase, to form a full-length double-stranded cDNA.

The random sequence primers used in the second strand synthesis reaction have a length that supports stable hybridization between the primers and a sequence that is 100% complementary thereto. The random sequence primers hybridize to a specific region in the first strand of the cDNA and permit DNA synthesis by primer extension from that region. In some embodiments, every possible nucleotide sequence is represented in the random sequence primer population or set. The primer size is generally kept short enough so that the all sequences can conveniently be represented in approximate equimolar quantities, but not so short that hybridization becomes unstable or indiscriminate. Generally a primer of the present invention comprises 5 nucleotides to 50 nucleotides, 6 nucleotides to 30 nucleotides, 7 nucleotides to 20 nucleotides, 7 nucleotides to 15 nucleotides, 7 nucleotides to 10 nucleotides, or 8 nucleotides to 9 nucleotides. The sets of random primers used for second strand synthesis may be of uniform or variable length. It should be noted that the term “random” as used with respect to the primers is not limited to primers selected in the mathematical sense of randomness. Random primer sequences may be selected on the basis of one or more criteria, for example, a predicted Tm, a predicted representation in the RNA population, and/or with consideration toward minimizing unwanted hybridization reactions and the like.

Nucleic Acid Amplification

The cDNA synthesis methods of the invention can be combined with nucleic acid amplification procedures to generate a useful pool of cDNA or cRNA molecules. In one embodiment, the invention provides a method for amplifying a population of mRNA molecules. Such a method involves synthesizing a pool of first cDNA strands as described herein, removing the template mRNA population, synthesizing a pool of double stranded cDNAs and then synthesizing an amplified RNA population.

To generate a pool of amplified cRNA molecules that reflect the diversity and relative molar ratios of the mRNA species in the mRNA population used for cDNA synthesis, an oligo dT-RNAP primer used in the initial reaction used for synthesizing the first cDNA strands. The oligo dT-RNAP primer is a pool of single stranded oligonucleotides, each having a first segment and a second segment, wherein the first segment comprises about 10 to about 30 dT nucleotides and the second segment comprises a recognition site for an RNA polymerase. Such an RNA Polymerase can be a SP6, T3 or T7 RNA polymerase.

RNA amplification may be performed by combining the cDNA template and an RNA polymerase, wherein the RNA polymerase can recognize an RNA promoter that is present in the cDNA template. Other reactants can be included such as ribonucleoside triphosphates. The reaction can include other components such as buffers and cofactors for the RNA polymerase. RNA amplification is then performed at a temperature and under conditions appropriate for RNA transcription by the selected RNA polymerase. A pyrophosphatase may be added to the reaction mixture in order to increase the yield of amplified product.

The methods of the invention can also be adapted to permit amplification by a thermostable DNA polymerase, for example, by polymerase chain reaction (PCR). Methods for PCR amplification are described in the art. For example, PCR Technology: Principles and Applications for DNA Amplification ed. H A Erlich, Stockton Press, New York, N.Y. (1989); PCR Protocols: A Guide to Methods and Applications, eds. Innis, Gelfland, Snisky, and White, Academic Press, San Diego, Calif. (1990); Mattila et al. (1991) Nucleic Acids Res. 19: 4967; Eckert, K. A. and Kunkel, T. A. (1991) PCR Methods and Applications 1: 17, each of which are incorporated herein by reference.

Kits

The invention further provides kits containing at least one container comprising at least one primer. The kit can contain other containers. For example, the kit can also contain another container comprising a second type of primer; the kit can contain another container comprising an enzyme such as a reverse transcriptase, DNA ligase or a DNA polymerase. The kit can contain other containers for useful reagents such as deoxyribonucleoside triphosphates (dNTPs), ribonucleoside triphosphates (NTPs), buffers, magnesium, and the like. The kit can also contain a means for purifying nucleic acids made using the kit, for example, a small column or spin column. The kits of the invention can also include instructions for using the primer(s) and other components of the kit. For example, the instructions can describe and teach one or more methods of the invention.

In one embodiment, the invention provides a kit for the preparation of cDNA from mRNA or a kit for amplifying a population of mRNA molecules. Such kits generally comprise a carrier that is compartmentalized to receive one or more containers in close confinement. The kit can include a first container that contains a primer for first strand cDNA synthesis, for example, a mixture of primers complementary to at least a segment of the template RNA population, oligo (dT) primers, oligo dT-RNAP primers or primers having SEQ ID NO:1. The oligo dT-RNAP primer encodes a promoter sequence for an RNA polymerase as well as being capable of hybridizing to a poly(A) tail of an mRNA. Hence, after cDNA synthesis the large amounts of cRNA can be made by use of an RNA Polymerase that recognizes and initiates transcription at the promoter. The kit can also include instructions for preparing cDNA by the methods of the invention.

The kit can include another container containing a mixture of primers having random nucleotide sequences. Such random primers are useful for second strand synthesis. Preferably, every possible nucleotide sequence is represented in the random sequence primers.

The kit can include another container containing a processive DNA polymerase, for example, Bacillus stearothermophilus DNA Polymerase in a known and convenient concentration for use in second strand synthesis.

The kit can further contain a container containing a reverse transcriptase having DNA polymerase activity or ligase. The kit can also contain a container containing a buffer. The kit can have another container with one or more deoxyribonucleoside triphosphates or ribonucleoside triphosphates. Containers with one or more labeled deoxyribonucleotides or ribonucleotides can also be provided in the kit. The kit can further contain a container containing a control RNA.

Each primer type may be provided in a separate container. For example, the first cDNA strand primer may be in a separate container from the second strand random sequence primers. The first cDNA strand primer is complementary to at least a portion of the RNA molecules in the template RNA population. An example of such a first cDNA strand primer is an oligo dT-RNAP primer, such as the primer having SEQ ID NO:1. The second strand cDNA primers can be a population of oligonucleotides having random nucleotide sequences as described in more detail above. When a mixture of primers is used (e.g. a mixture of primers with random sequences), each primer type may be present in any convenient stoichiometry. In general, each primer type in a mixture of primers is present in approximately equimolar concentrations.

Moreover, primers may be provided that are attached to a solid support. Primers may be partially double stranded, particularly when attached to a solid support. Primers may be covalently coupled to a solid support using standard chemical procedures. One such method of coupling incorporates linker-arms in the complementary strand, where the linker-arm is modified to react primarily with a functional moiety.

The polynucleotide primers may be prepared using any suitable method, such as, for example, the phosphotriester and phosphodiester methods, or automated embodiments thereof. In one such automated embodiment diethylphosphoramidites are used as starting materials and may be synthesized as described by Beaucage et al. (1981) Tetrahedron Letters 22: 1859. One method for synthesizing oligonucleotides on a modified solid support is described in U.S. Pat. No. 4,458,066. It is also possible to use a primer that has been isolated from a biological source, such as a restriction endonuclease digest or the like. Methods for preparing and using probes and primers are described in the references, for example Sambrook et al., 1989, Molecular Cloning: A Laboratory Manual, 2nd ed., vol. 1-3, Cold Spring Harbor Press, Plainview N.Y.; Ausubel et al., 1987, Current Protocols in Molecular Biology, Greene Publ. Assoc. & Wiley-Intersciences, New York N.Y.; Innis et al., 1990, PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

It is to be understood that this invention is not limited to particular methods and kits described, as such methods and kits may, of course, vary. It is also to be understood that the terminology used herein is for the purpose of describing particular embodiments only, and is not intended to be limiting, since the scope of the present invention will be limited only by the appended claims.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperatures, etc.) but some experimental errors and deviations should be accounted for.

### EXAMPLE 1

### Initial Testing of cDNA Synthesis by Random Priming

In this Example, initial testing of randomly primed cDNA synthesis was compared to cDNA synthesis using RNase H.

Amplification reactions were performed using a single mRNA as the target mRNA to be amplified, ATP5F1 (Acc #: AA453849, purchased from the American Type Culture Collection (ATCC)). The target was amplified by PCR using a pair of primers to generate an approximate 600 bp double stranded DNA that contains both a T7 promoter and a T3 promoter. Complementary RNA (cRNA) was generated from this 600 bp ATP5F1 DNA using the T7 promoter and T7 RNA polymerase. The cRNA (0.5 μg/μl) was isolated and then used as template for cDNA synthesis reactions.

Conditions for random priming of the second cDNA strand were not optimized in a first reaction that was used to compare random priming with the RNase H method of second strand synthesis. For first strand synthesis, two identical reaction mixtures were set up. Each first strand reaction mixture contained 4 μl ATP5F1 cRNA template (2 μg) mixed with 4 μl water and 2 μl of 10 μM T7-dT24 primer having SEQ ID NO:1 (CGAATTTAATACGACTCACTATAGGGAGATTTTTTTTTTTTTTTTTTTTTT TT). A control primer having the sequence (dT)₂₁ (SEQ ID NO:2) was also employed in some reactions. Each reaction mixture was heated to 70° C. for 10 min. and then chilled on ice. The separate mixtures were then mixed with 4 μl of 5×1^{st} strand synthesis buffer, 2 μl of 100 mM DTT, 1 μl of 10 mM dNTPs, 1 μl of 20 U/μl of RNase Inhibitor and 2 μl of MuLV reverse transcriptase to generate a 20 μl 1^{st} strand reaction mixture. These reaction mixtures were incubated at 40° C. for 2 hr and then heat inactivated at 65° C. for 15 min. A second first strand synthesis reaction was also performed using Superscript II reverse transcriptase under similar conditions.

After chilling on ice, two different second strand synthesis reaction mixtures were set up.

For second strand synthesis by the RNase H method, a second strand synthesis reaction mixture was prepared containing 91 μl of water, 30 μl of 5×2^{nd} strand synthesis buffer, 3 μl of 10 mM dNTPs, 1 μl of 10 U/μl of E. coli DNA Ligase, 4 μl of 10 U/μl E. coli DNA Polymerase 1 and 1 pl of 2 U/μl RNase H. Fifteen μl of the reverse transcription (first strand synthesis) reaction was combined with 5 μl of water and then added to the second strand synthesis reaction mixture. This mixture was then incubated at 16° C. for 2 hr, heat inactivated at 65° C. for 15 min and then chilled on ice.

For second strand synthesis by the random priming method, a second strand synthesis reaction mixture was prepared containing 6 μl of water, 10 μl of 6-9 nucleotide random primer solution, 5 μl of 10 mM dNTPs, 5 pl 10×T4 Ligase Buffer, 2.5 μl of T4 DNA Ligase, and 1.5 μl of the Klenow fragment of DNA Polymerase I. Twenty μl of the reverse transcription (first strand synthesis) reaction was combined with this second strand synthesis reaction mixture. This mixture was then incubated at 20° C. for 2 hr, heat inactivated at 70° C. for 10 min and then chilled on ice.

The cDNA products generated by the RNase H and random priming methods were separately purified by use of a QIAquick Spin Column (QIAGEN), amplified by in vitro transcription using Ambion's T7 Kit and then separated on a 2% agarose gel.

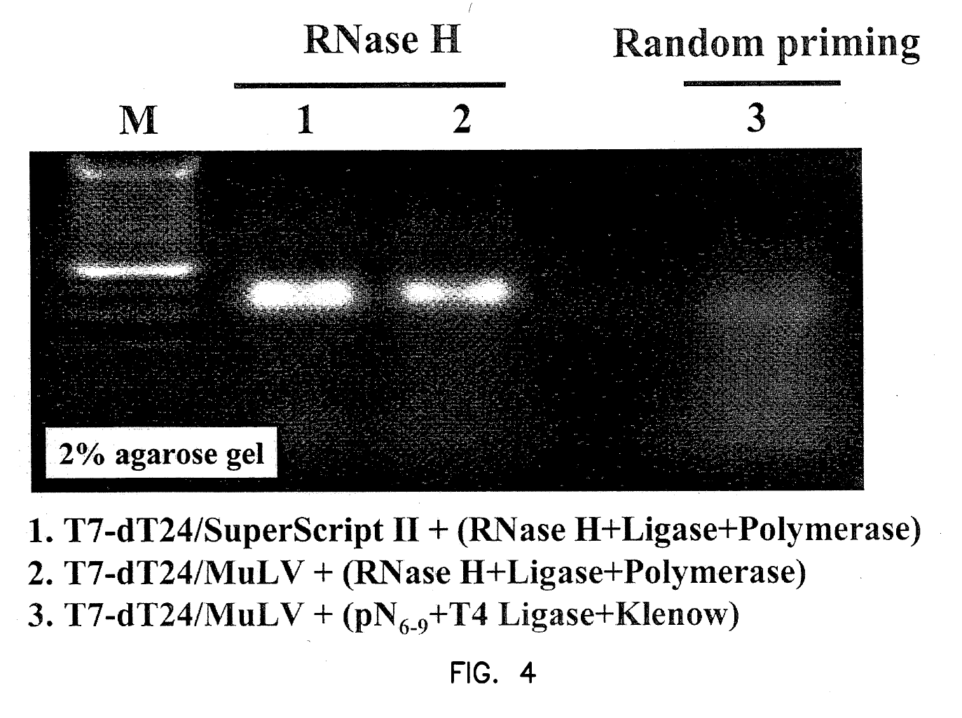

Results of this experiment are provided in FIG. 4, which illustrates that, under the conditions tested, the RNase H method produced more full-length ATP5F1 cDNA than the random priming reaction.

### EXAMPLE 2

### Optimization of Randomly Primed cDNA Synthesis

In this Example, randomly primed cDNA synthesis was performed under a variety of conditions to optimize second strand synthesis.

Amplification reactions were performed using a single mRNA as the target mRNA to be amplified, ATP5F1 (Acc #: AA453849, purchased from the American Type Culture Collection (ATCC)). The target was amplified by PCR using a pair of primers to generate an approximate 600 bp double stranded DNA that contains both a T7 promoter and a T3 promoter. Complementary RNA (cRNA) was generated from this 600 bp ATP5F1 DNA using the T7 promoter and T7 RNA polymerase. The cRNA (0.5 μg/ml) was isolated and then used as template for cDNA synthesis reactions.

For first strand synthesis, two identical reaction mixtures were set up. Each first strand reaction mixture contained 4 μl ATP5F1 cRNA template (2 μg) mixed with 4 μl water and 2 μl of 10 μM T7-dT24 primer having SEQ ID NO:1 (CGAATTTAATACGACTCACTATAGGGAGATTTTTTTTTTTTTTTTTTTTTT TT). A control primer having the sequence (dT)₂₁ (SEQ ID NO:2) was also employed in some reactions. Each reaction mixture as heated to 70° C. for 10 min. and then chilled on ice. The separate mixtures were then mixed with 4 μl of 5×1^{st} strand synthesis buffer, 2 μl of 100 mM DTT, 1 μl of 10 mM dNTPs, 1 μl of 20 U/μl of RNase Inhibitor and 2 μl of MuLV reverse transcriptase to generate a 20 μl 1^{st} strand reaction mixture. These reaction mixtures were incubated at 42° C. for 2 hr and then heat inactivated at 65° C. for 15 min. A second first strand synthesis reaction was also performed using Superscript II reverse transcriptase under similar conditions.

After chilling on ice, two different second strand synthesis reaction mixtures were set up.

For second strand synthesis by the RNase H method, a second strand synthesis reaction mixture was prepared containing 91 μl of water, 30 μl of 5×2^{nd} strand synthesis buffer, 3 μl of 10 mM dNTPs, 1 μl of 10 U/μl of E. coli DNA Ligase, 4 μl of 10 U/μl E. coli DNA Polymerase 1 and 1 μl of 2 U/μl RNase H. Twenty μl of the reverse transcription (first strand synthesis) reaction was added to the second strand synthesis reaction mixture. This mixture was then incubated at 16° C. for 2 hr. Two microliters of 5 U/μl T4 DNA polymerase was added. The reaction continued to incubate at 16° C. for 15 minutes.

For second strand synthesis by the random priming method, the RNA template was first removed by alkaline hydrolysis by adding 3 μl of 2.5 M NaOH to the first strand reaction mixture. The NaOH and excess primer were removed by Microcon-100 filtration column purification. A second strand synthesis reaction mixture was prepared containing 3.5 μl of water, 10 μl of 100 μM 6-9 nucleotide random primers, 2.5 μl of 10 mM dNTPs, 5 μl 10× ligase or polymerase buffer, 2.5 μl of T4 DNA Ligase, and 1.5 μl of a selected DNA polymerase. A variety of DNA polymerases were tested including Klenow, Klenow (exo-), T4 DNA polymerase, T7 DNA polymerase and E. coli DNA Polymerase I. Twenty-five μl of the purified reverse transcription (first strand synthesis) reaction was combined with this second strand synthesis reaction mixture. This mixture was then incubated at a variety of incubation temperatures for 2 hr. The incubation temperatures tested included 20° C., 25° C. and 37° C. After incubation, the reaction was heat inactivated at 70° C. for 10 min and then chilled on ice.

The cDNA products generated by the RNase H and random priming methods were separately purified by use of a QIAquick Spin Column (QIAGEN), amplified by in vitro transcription using Ambion's T7 Kit and then separated on a 2% agarose gel.

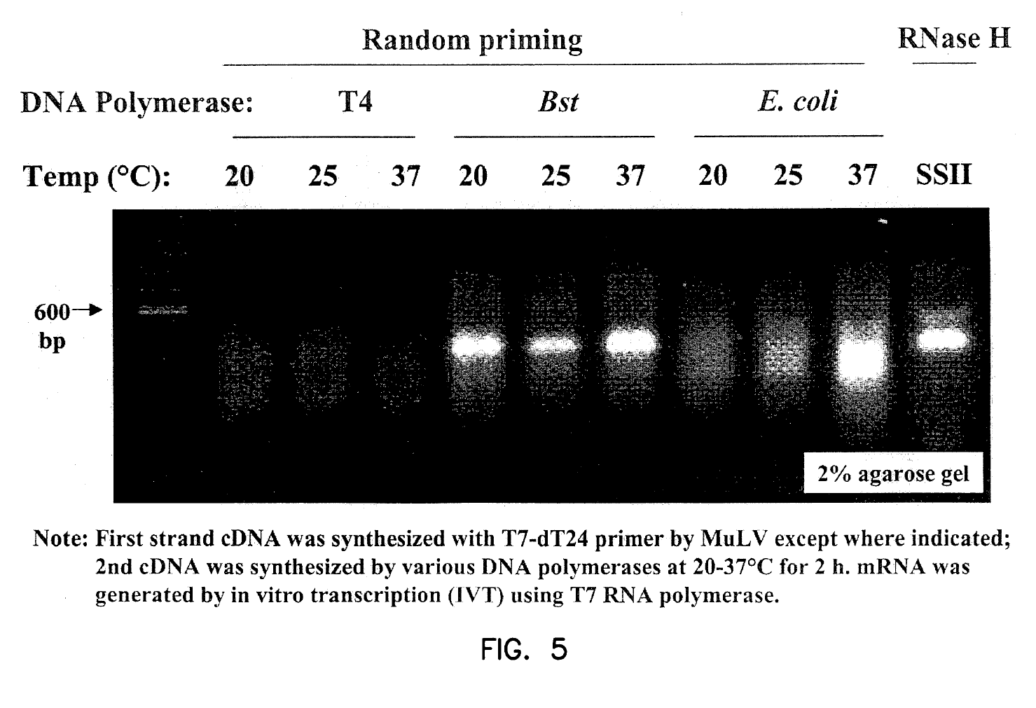

FIG. 5 illustrates that Bacillus stearothermophilus (Bst) DNA polymerase is the best DNA polymerase and 37° C. is the best incubation temperature for use in the random priming reaction. While not systematically tested it appeared that slightly longer primers (e.g., 8-9 nucleotides in length) provided somewhat better second strand synthesis than shorter primers (e.g., 6-7 nucleotides in length). The quantity and quality of amplified ATP5F1 cRNA produced using Bst DNA polymerase at 37° C. was comparable to that produced using the RNase H method (FIG. 5, Table 1).

TABLE 1
RNA Yield for RNase H vs. Random Priming Methods

| cDNA Synthesis | | Incubation | RNA Yield |
|---|---|---|---|
| Method | DNA Polymerase | Temperature (° C.) | (μg) |
| RNase H | E. coli | 20 | 79 |
| Random Priming | T4 | 20 | 29 |
| Random Priming | T4 | 25 | 28 |
| Random Priming | T4 | 37 | 18 |
| Random Priming | Bst | 20 | 67 |
| Random Priming | Bst | 25 | 74 |
| Random Priming | Bst | 37 | 76 |

### EXAMPLE 3

### The Randomly Primed cDNA Synthesis Method Generates Low and High Abundance mRNAs

In this Example, the optimized randomly primed cDNA synthesis method was performed using several different mRNA populations to test whether the method selectively amplified one type of mRNA molecule over another.

1^{st} Strand cDNA Synthesis

Total human liver mRNA was purchased from Ambion and synthetically pooled mRNA was generated from cloned genes using the MEGAScript T7 Kit from Ambion. Six tubes containing the following types and amounts of template mRNA were prepared for annealing.

TABLE 2
First Strand Annealing Mixture

| | | Primer | | |
|---|---|---|---|---|
| Tube | mRNA | (SEQ ID NO: 1) | Water | Total |
| 1 | ATP5F1 (2 μg) | 3 μl | 8 μl | 15 μl |
| 2 | Pool I (2 μg) | 3 μl | 8 μl | 15 μl |
| 3 | Pool II (2 μg) | 3 μl | 8 μl | 15 μl |
| 4 | Pool II (0.2 μg) | 3 μl | 11.8 μl | 15 μl |
| 5 | Human mRNA (1 μg) | 3 μl | 11 μl | 15 μl |
| 6 | Human mRNA (10 μg) | 3 μl | 2 μl | 15 μl |

The types and molar ratios of the various RNAs in pools I, II and III that were used as template RNAs for these experiments are provided in Table 3.

TABLE 3
Ratios of mRNAs in Starting Pools

| Gene | Pool I | Pool II | Pool III |
|---|---|---|---|
| ATP5B | 1 | 1 | 1 |
| ATP5F1 | 1 | 1 | 1 |
| BRCA1 | 1 | 0.05 | 0.01 |
| CETP | 1 | 0.05 | 0.01 |
| COX6B | 1 | 0.05 | 0.01 |
| EIFIA | 1 | 0.05 | 0.01 |
| PRKCA | 1 | 0.05 | 0.01 |
| RPS4X | 1 | 0.01 | 0.001 |
| PEX7 | 1 | 0.2 | 0.1 |
| APOC2 | 1 | 0.2 | 0.1 |
| ARF3 | 1 | 0.01 | 0.001 |
| ATP7A | 1 | 1 | 1 |
| PPP1CA | 1 | 0.2 | 0.1 |
| G6PD | 1 | 1 | 1 |
| ODC1 | 1 | 0.2 | 0.1 |
| ATP6B2 | 1 | 0.2 | 0.1 |
| PLAT | 1 | 0.01 | 0.001 |
| JUNB | 1 | 0.01 | 0.001 |
| ATP6E | 1 | 1 | 1 |
| MYL2 | 1 | 0.01 | 0.001 |

The mRNA template was mixed with 5 μM T7-dT24 primer (CGAATTTAATACGACTCACTATAGGGAGATTTTTTTTTTTTTTTTTTTTTT TT, SEQ ID NO:1) and denatured at 70° C. for 5 min. A control primer having the sequence (dT)₂₁ (SEQ ID NO:2) was also employed in some reactions. First strand cDNA synthesis was performed using 100-200 Units MuLV reverse transcriptase, 1 mM dNTPs and 30 Units RNase inhibitor in IX RT buffer from Applied Biosystems cDNA Archiving Kit. The reaction was performed at 42° C. for 2 h. The enzyme was inactivated by heating at 65° C. for 15 min. The RNA template was removed by alkaline hydrolysis by adding 3 μl of 2.5 M NaOH to the first strand reaction mixture. The NaOH and excess primer was removed by Microcon-100 filtration column purification.

2nd Strand cDNA Synthesis

The second strand of the cDNA preparations was synthesized using 10 μM of 5′ phosphorylated random oligonucleotide primers (8-9 nucleotides in length), 0.1-1 U/ul Bst DNA polymerase and 16 Units/μl T4 DNA ligase at 37° C. for 2 h. The cDNA pool was made blunt-ended by treatment with 10-20 Units of T4 DNA polymerase for 15 min at 37° C. Double-stranded cDNA was purified by filtration column (Microcon-100) or affinity capture column (QIAGEN's QIAquik).

In Vitro Transcription to Generate Labeled, Amplified cRNA

Double-stranded cDNA containing T7 RNA polymerase recognition sites was transcribed at 37° C. for 6 to 9 h in 20 μl reaction mixtures containing 10-40 Units/μl T7 RNA polymerase, 7.5 mM each of ATP, CTP and GTP, 7-3 mM UTP, 0.5-2.5 mM Dye-linker-UTP, 20 mM MgCl₂, 40 mM Tris-HCl, pH 8.0, 10 mM DTT, and 2 mM spermidine. Amplified cRNA was purified with QIAGEN's RNeasy column to remove unincorporated dye-labeled UTP.

The yield of cRNA was measured by absorbance readings on a 1:50 dilution of the cRNA product using the following equation to calculate cRNA concentration.

RNA yield (μg/μl)=(A₂₆₀−A₃₂₀)×50×(40/1000)

Table 4 provides the yield of cRNA obtained using the different mRNA templates.

TABLE 4
cRNA Yield

| | Amount mRNA | Yield: RNase H | Yield: Random |
|---|---|---|---|
| Sample | Template (μg) | Method (μg) | Priming (μg) |
| ATP5F1 | 0.16 | 77 | 98 |
| Pool I | 0.16 | 76 | 109 |
| Pool II | 0.16 | 84 | 105 |
| Pool II | 0.016 | 14 | 18 |
| Human mRNA | 0.2 | 16 | 46 |
| Human mRNA | 2.0 | 2 | 27 |

As illustrated by Table 4, the random priming method produced more cRNA than did the RNase H method even when complex human RNA was used as template.

The quality and relative yield of the different mRNAs was assessed by purification of the cRNA and visualization on a 2% agarose gel (FIG. 6). FIG. 6 shows that each cRNA product was substantially full length. These results were confirmed by further tests and gel analyses (results not shown). Moreover, amplification of pooled mRNA templates having different molar ratios of the different mRNA types (as described in Table 3), generated relatively the same ratio of each cRNA product in the amplified pools, as assessed by observation of the density of individual bands on gels and as confirmed by PCR analysis. Hence, the random primer method for second strand cDNA synthesis generated substantially full-length cDNA products and exhibits substantially no tendency to discriminate between low and high abundance mRNA templates.

### REFERENCES

Baugh, L. R., A. A. Hill, et al. (2001). “Quantitative analysis of mRNA amplification by in vitro transcription.” Nucleic Acids Res 29(5): E29.

Gubler, U. and B. J. Hoffman (1983). “A simple and very efficient method for generating cDNA libraries.” Gene 25(2-3): 263-9.

Kotewicz, M. L., C. M. Sampson, et al. (1988). “Isolation of cloned Moloney murine leukemia virus reverse transcriptase lacking ribonuclease H activity.” Nucleic Acids Res 16(1): 265-77.

Land, H., M. Grez, et al. (1981). “5′-Terminal sequences of eucaryotic mRNA can be cloned with high efficiency.” Nucleic Acids Res 9(10): 2251-66.

Lockhart, D. J. and E. A. Winzeler (2000). “Genomics, gene expression and DNA arrays.” Nature 405(6788): 827-36.

Mahadevappa, M. and J. A. Warrington (1999). “A high-density probe array sample preparation method using 10- to 100-fold fewer cells.” Nat Biotechnol 17(11): 1134-6.

Okayama, H. and P. Berg (1982). “High-efficiency cloning of full-length cDNA.” Mol Cell Biol 2(2): 161-70.

Okayama, H. and P. Berg (1992). “High-efficiency cloning of full-length cDNA. 1982.” Biotechnology 24: 210-9.

Van Gelder, R. N., M. E. von Zastrow, et al. (1990). “Amplified RNA synthesized from limited quantities of heterogeneous cDNA.” Proc Nat'l Acad Sci USA 87(5): 1663-7.

All publications mentioned herein are incorporated herein by reference to disclose and describe the methods and/or materials in connection with which the publications are cited.

What is claimed:

1. A method for synthesizing a double-stranded cDNA comprising:
(a) synthesizing a pool of first cDNA strands in a first reaction mixture comprising reverse transcriptase, an RNA template and a first strand primer that is complementary to a least one RNA template;
(b) removing the RNA template; and
(c) synthesizing a pool of double stranded cDNAs in a first reaction mixture comprising a processive DNA polymerase, a DNA ligase, a pool of first cDNA strands as template and multiple random primers,
wherein the multiple random primers comprise a mixture of oligonucleotides having random DNA sequences.

2. The method of claim 1, wherein the first strand primer is an oligo dT-RNAP primer comprising a single stranded oligonucleotide with a first segment and a second segment, and wherein the first segment comprises about 10 to about 30 deoxyribothymidine nucleotides and the second segment comprises a recognition site for an RNA polymerase.

3. The method of claim 2, wherein the RNA polymerase is T7 RNA polymerase, T3 RNA polymerase or SP6 RNA polymerase.

4. The method of claim 2, wherein the oligo dT-RNAP primer comprises SEQ ID NO:1.

5. The method of claim 1, wherein the reverse transcriptase is moloney murine leukemia virus reverse transcriptase.

6. The method of claim 1, wherein the first reaction mixture further comprises a mixture of deoxyribonucleoside triphosphates.

7. The method of claim 1, wherein the second reaction mixture further comprises a mixture of deoxyribonucleoside triphosphates.

8. The method of claim 1, wherein the processive DNA polymerase has strand displacement activity.

9. The method of claim 1, wherein the processive DNA polymerase does not have substantial 3′ to 5′ exonuclease activity.

10. The method of claim 1, wherein the processive DNA polymerase is a Bacillus stearothermophilus DNA polymerase.

11. A method for amplifying a population of RNA molecules, comprising:
(a) synthesizing a pool of first cDNA strands in a first reaction mixture comprising reverse transcriptase, a template RNA population, and a first strand primer, to produce a pool of first cDNA strands;
(b) removing the template RNA population;
(c) synthesizing a pool of double stranded cDNAs in a second reaction mixture comprising the pool of first cDNA strands as template, a processive DNA polymerase, a DNA ligase, and multiple random primers, to produce a pool of double-stranded cDNA molecules;
(d) synthesizing an amplified complementary RNA population in a third reaction mixture comprising an RNA polymerase and the pool of double-stranded cDNA molecules as template, to produce an amplified complementary RNA population having about the same proportion of each type of complementary RNA as in the template RNA population of step (a);
wherein the first strand primer comprises a pool of single stranded oligonucleotides each having a first segment and a second segment, wherein the first segment comprises about 10 to about 30 nucleotides that are complementary to at least a portion of RNA molecules in the template RNA population and the second segment comprises a recognition site for a RNA polymerase.

12. The method of claim 11, wherein the template RNA population comprises at least one low abundance mRNA and at least one high abundance mRNA.

13. The method of claim 11, wherein the processive DNA polymerase has strand displacement activity.

14. The method of claim 11, wherein the processive DNA polymerase does not have substantial 3′ to 5′ exonuclease activity.

15. The method of claim 11, wherein the processive DNA polymerase is a Bacillus stearothermophilus DNA polymerase.

16. The method of claim 11, wherein the RNA polymerase is T7 RNA polymerase, T3 RNA polymerase or SP6 RNA polymerase.

17. The method of claim 11, wherein the first strand primer comprises an oligo dT-RNAP primer that comprises SEQ ID NO:1.

18. The method of claim 11, wherein the reverse transcriptase is moloney murine leukemia virus reverse transcriptase.

19. The method of claim 11, wherein the first reaction mixture further comprises a mixture of deoxyribonucleoside triphosphates.

20. The method of claim 11, wherein the second reaction mixture further comprises a mixture of deoxyribonucleoside triphosphates.

21. A kit for synthesizing a double-stranded cDNA comprising a first container containing a processive DNA polymerase and a second container containing multiple random primers, wherein the multiple random primers comprise a mixture of oligonucleotides having random DNA sequences.

22. The kit of claim 21, that further comprises a third container containing oligo dT-RNAP primers, wherein the oligo dT-RNAP primers comprise a pool of single stranded oligonucleotides each having a first segment and a second segment, wherein the first segment comprises about 10 to about 30 deoxyribothymidine nucleotides and the second segment comprises a recognition site for a RNA polymerase.

23. The kit of claim 21, wherein the RNA polymerase is T7 RNA polymerase, T3 RNA polymerase or SP6 RNA polymerase.

24. The kit of claim 22, wherein the oligo dT-RNAP primer comprises SEQ ID NO:1.

25. The kit of claim 21 that further comprises a container containing a reverse transcriptase.

26. The kit of claim 21 that further comprises a container containing a mixture of deoxyribonucleoside triphosphates.

27. The kit of claim 21 that further comprises a container containing a mixture of ribonucleoside triphosphates.

28. The kit of claim 21 that further comprises a container containing at least one deoxyribonucleoside triphosphate conjugated to a detectable label.

29. The kit of claim 21 that further comprises a container containing at least one ribonucleoside triphosphate conjugated to a detectable label.

30. The kit of claim 21 that further comprises a container containing a DNA ligase.

31. The kit of claim 21 that further comprises a container containing a buffer solution.

32. The kit of claim 21 that further comprises instructions for synthesizing a double-stranded cDNA.

33. The kit of claim 21, wherein the processive DNA polymerase has strand displacement activity.

34. The kit of claim 21, wherein the processive DNA polymerase does not have substantial 3′ to 5′ exonuclease activity.

35. The kit of claim 21, wherein the processive DNA polymerase is a Bacillus stearothermophilus DNA polymerase.

36. A kit for amplifying a population of mRNA molecules, comprising a first container containing a processive DNA polymerase, a second container containing multiple random primers, wherein the multiple random primers comprise a mixture of oligonucleotides having random DNA sequences; and a third container containing oligo dT-RNAP primers, wherein the oligo dT-RNAP primers comprise a pool of single stranded oligonucleotides each having a first segment and a second segment, wherein the first segment comprises about 10 to about 30 deoxythymidine nucleotides and the second segment comprises a recognition site for a RNA polymerase.

37. The kit of claim 36, wherein the RNA polymerase is T7 RNA polymerase, T3 RNA polymerase or SP6 RNA polymerase.

38. The kit of claim 36, wherein the oligo dT-RNAP primers comprise SEQ ID NO:1.

39. The kit of claim 36 that further comprises a container containing a reverse transcriptase.

40. The kit of claim 36 that comprises a container containing a DNA ligase

41. The kit of claim 36 that further comprises a container containing a mixture of deoxyribonucleoside triphosphates.

42. The kit of claim 36 that further comprises a container containing a mixture of ribonucleoside triphosphates.

43. The kit of claim 36 that further comprises a container containing at least one deoxyribonucleoside triphosphate conjugated to a detectable label.

44. The kit of claim 36 that further comprises a container containing at least one ribonucleoside triphosphate conjugated to a detectable label.

45. The kit of claim 36 that further comprises instructions for amplifying a population of mRNA molecules.

46. The kit of claim 36, wherein the processive DNA polymerase has strand displacement activity.

47. The kit of claim 36, wherein the processive DNA polymerase does not have substantial 3′ to 5′ exonuclease activity.

48. The kit of claim 36, wherein the processive DNA polymerase is a Bacillus stearothermophilus DNA polymerase.


SEQUENCE LISTING (2 sequences)

SEQ ID NO: 1 - DNA, 53 bp, Artificial Sequence
  A dT-RNAP primer.
cgaatttaatacgactcactatagggagatttttttttttttttttttttttt

SEQ ID NO: 2 - DNA, 21 bp, Artificial Sequence
  A control primer.
ttttttttttttttttttttt